(19) United States
(12) Patent Application Publication
(10) Pub. No.: US 2023/0346288 A1
(43) Pub. Date: Nov. 2, 2023

(54) Systems and Methods for Evaluating Cardiovascular Disease Risks

(71) Applicant: The Board of Trustees of the Leland Stanford Junior University, Stanford, CA (US)

(72) Inventors: John Weston Hughes, Stanford, CA (US); James Zou, Stanford, CA (US); Euan A. Ashley, Stanford, CA (US); Marco V. Perez, San Jose, CA (US)

(73) Assignee: The Board of Trustees of the Leland Stanford Junior University, Stanford, CA (US)

(21) Appl. No.: 18/309,648

(22) Filed: Apr. 28, 2023

Related U.S. Application Data
(60) Provisional application No. 63/363,802, filed on Apr. 28, 2022.

(51) Int. Cl. A61B 5/346; A61B 5/00; G16H 50/30

(52) U.S. Cl. CPC A61B 5/346; A61B 5/7267; G16H 50/30; A61B 5/0006

(57) ABSTRACT

Systems and methods for predicting a future cardiovascular event are provided. Electrocardiogram waveform data can be acquired and utilized in a trained computational model to predict a future cardiovascular event. Clinical interventions, clinical surveillance, and clinical treatments can be performed based on a future cardiovascular event prediction.

### CROSS REFERENCE TO RELATED APPLICATIONS

This application claims priority to U.S. Provisional Application Ser. No. 63/363,802, entitled “Systems and Methods for Evaluating Cardiovascular Disease Risks,” filed Apr. 28, 2022, which is incorporated herein by reference in its entirety.

### STATEMENT REGARDING FEDERALLY SPONSORED RESEARCH OR DEVELOPMENT

This invention was made with Government support under contract DGE-1656518 awarded by the National Science Foundation and under contract 1942926 awarded by the National Science Foundation. The Government has certain rights in the invention.

### TECHNOLOGICAL FIELD

The disclosure is generally directed to systems and methods to assess cardiovascular disease risks using electrocardiogram data.

### BACKGROUND

Cardiovascular disease remains the most common cause of death in the United States and globally, despite the availability of statins and other therapies. These treatments are prescribed according to the concept that patients with greater risk should receive greater care, making accurate risk stratification valuable. Even so, commonly used risk scores like the pooled cohort equations (PCE) for prediction of atherosclerotic cardiovascular disease (ASCVD) suffer from middling accuracy and miscalibration and make use of only a short list of simple risk factors.

Electrocardiograms (ECGs) are the most frequently used cardiovascular diagnostic tool for near-term cardiovascular events. Abnormalities in the electrocardiogram indicate higher cardiovascular and all-cause mortality and with higher incidence of major cardiovascular events within one year.

### SUMMARY

Several embodiments are directed to systems and methods to predict risk of cardiovascular disease and/or mortality using ECG data. In many embodiments, a trained computational model is utilized to evaluate cardiovascular disease and/or mortality risk. In several embodiments, the cardiovascular disease and/or mortality risk is predicted over a prolonged timeline. In many embodiments, a prediction of cardiovascular disease and/or mortality risk is utilized to determine clinical intervention and/or treatments. In some embodiments, ECG data and the trained computational model is utilized to augment and/or stratify predictions generated by pooled cohort equations.

In some implementations, a computational method is for predicting a future cardiovascular event. The method comprises obtaining, using a computational processing system, electrocardiogram data derived from an individual. The electrocardiogram waveform comprises one or more electrocardiogram waveforms. The method comprises predicting, using the computational processing system and a trained computational model, a risk that the individual will experience a cardiovascular disease event in the future. The trained computational model utilizes the one or more electrocardiogram waveforms to predict the likelihood of the cardiovascular disease event.

In some implementations, the trained computational model is trained from electrocardiogram data obtained from a cohort of individuals having cardiovascular health records that include a timeline of cardiovascular events after collection of each individual's electrocardiogram data.

In some implementations, the computational model is a deep neural network (DNN), a convolutional neural network (CNN), a recurrent neural network, a long short-term memory (LSTM) network, a kernel ridge regression, or a gradient-boosted random forest decision tree.

In some implementations, the trained computational model predicts the risk that the individual will experience a cardiovascular disease event that will occur in more than one year.

In some implementations, the trained computational model predicts the risk that the individual will experience a cardiovascular disease event that will occur within five years.

In some implementations, the trained computational model predicts the risk that the individual will experience a cardiovascular disease event that will occur within ten years.

In some implementations, the cardiovascular event is development of atherosclerotic cardiovascular disease (ASCVD), infarction, heart failure, non-lethal heart attack, lethal heart attack, stroke, sudden cardiac death, or a combination thereof.

In some implementations, the cardiovascular event is development of atherosclerotic cardiovascular disease (ASCVD). The method further comprises estimating, using the computational processor, a risk of ASCVD of the individual via the pooled cohort equation (PCE). The method further comprises combining, using the computational processor, the estimated risk of ASCVD as estimated by PCE risk with the predicted likelihood that the individual is to develop ASCVD as determined by the trained computational model to yield a combined risk assessment.

In some implementations, the method further comprises administering a statin to the individual, wherein the individual was estimated to be a low risk of developing ASCVD by PCE and high risk of developing ASCVD by the trained computational model.

In some implementations, the method further comprises halting the administering of a statin to the individual, wherein the individual was estimated to be a high risk of developing ASCVD by PCE and low risk of developing ASCVD by the trained computational model.

In some implementations, the method further comprises performing a clinical intervention, clinical monitoring, or a treatment based on a future cardiovascular disease event prediction.

In some implementations, the method further comprises acquiring, using a set of one or more leads of an electrocardiogram, electrocardiogram data of the individual. The method further comprises generating, using a computational processor, the one or more electrocardiogram waveforms utilized within the computational model to predict the risk of that the individual will experience a cardiovascular disease event in the future.

In some implementations, an electrocardiogram system is for predicting future cardiovascular events of patients. The system comprises an electrocardiogram device comprising a set of one or more leads capable of acquiring electrical signals of an individual. The system comprises a computational processing system in communication with the electrocardiogram device. The computational processing system comprises a memory comprising an application for performing an electrocardiogram and an application comprising a trained computational model for predicting future cardiovascular events. The computational processing system comprises a processor. The application for performing an electrocardiogram directs the processor to collect electrical signals of an individual and generate electrocardiogram data. The electrocardiogram data comprises a set of one or more electrocardiogram waveforms. The application comprising a trained computational model for predicting future cardiovascular events directs the processor to obtain the electrocardiogram data and predict the risk that an individual will experience a cardiovascular disease event in the future utilizing the set of one or more electrocardiogram waveforms.

In some implementations, the trained computational model is trained from electrocardiogram data obtained from a cohort of individuals having cardiovascular health records that include a timeline of cardiovascular events after collection of each individual's electrocardiogram data.

In some implementations, the trained computational model predicts the risk that the individual will experience a cardiovascular disease event that will occur in more than one year.

In some implementations, the trained computational model predicts the risk that the individual will experience a cardiovascular disease event that will occur within five years.

In some implementations, the cardiovascular event is development of atherosclerotic cardiovascular disease (ASCVD), infarction, heart failure, non-lethal heart attack, lethal heart attack, stroke, sudden cardiac death, or a combination thereof.

In some implementations, the computational processing system is housed within a computing device that is in direct association the electrocardiogram device.

In some implementations, the computational processing system is housed within a computing device that is separate of the electrocardiogram device and obtains the electrocardiogram data via a wireless connection.

In some implementations, the electrocardiogram device and the computational processing system is housed within a wearable device.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow diagram of a method to predict a cardiovascular disease event in accordance with various embodiments.

FIG. 2 provides an example of comparing cardiovascular disease event prediction results of three ECG waveforms of patients with similar traditional risk factors.

FIG. 3 provides an example of combining pooled cohort equations with a computational model for prediction of cardiovascular disease to better stratify.

FIG. 4 provides a conceptual illustration of a computational processing system in accordance with various embodiments.

FIG. 5 provides a table describing performance of Stanford Estimator of Electrocardiogram Risk (SEER) on three test sets, among all patients.

FIGS. 6A and 6B provides Receiver Operator Characteristic (ROC) curves and Areas Under the Curve (AUCs) for Stanford, Cedars-Sinai, and Columbia test sets; Cumulative incidence of cardiovascular mortality in the Stanford PCE comparison set (Kaplan-Meier estimates); and Hazard ratios of various cardiovascular diseases given that a patient is in the top third of SEER risk, in the Stanford cross-validation set.

FIGS. 7A and 7B provides cumulative incidence of atherosclerotic cardiovascular disease in the Stanford PCE comparison set (Kaplan-Meier estimates), among patients called low-risk (0-7.5%) moderate risk (7.5%-20%), and high-risk (20-100%) by the PCE. The dotted line shows the 7.5% risk cutoff used in the decision to prescribe statins.

FIG. 8 provides a table describing performance of SEER and the PCE Score on the SUMC cross-validation comparison set (non-inpatients who had never suffered a prior cardiovascular disease event, were non-diabetic, and had an LDL cholesterol measurement below 190 mg/dL and any blood pressure measurement within the year prior to the ECG).

FIG. 9 provides 10-year incidence of atherosclerotic cardiovascular disease in different groups (Kaplan-Meier estimates) in the Stanford PCE comparison set, with group counts.

FIG. 10 provides a table on performance on different protected groups.

FIGS. 11A to 11C provide cumulative incidence of atherosclerotic cardiovascular disease in the Stanford PCE comparison set (Kaplan-Meier estimates), among patients in various demographic groups. The dotted line shows the 7.5% risk cutoff used in the decision to prescribe statins.

FIG. 12A provides age and sex-corrected odds ratios for being in the top third of SEER risk, given each diagnosis present in the electrocardiogram cardiologist overread, in the Stanford cross-validation set.

FIG. 12B provides age and sex-corrected odds ratios for falling in each bin of three continuous variables (age is not age-corrected).

FIG. 12C provides examples from 8 different patients from the bottom third of SEER risk, lead I (top panel) and examples from 8 different patients from the top third of SEER risk, lead I (bottom panel).

FIG. 13 provides a list of features for random forest models.

FIG. 14 provides a table providing data on performance of random forest baselines on the Stanford cross-validation set in predicting 5-year cardiovascular mortality.

FIG. 15 provides a diagram of a model.

FIG. 16 provides 10-year incidence of cardiovascular mortality in different groups (Kaplan-Meier estimates) in the Stanford PCE comparison set, with group counts.

### DETAILED DESCRIPTION

Turning now to the drawings and data, various systems and methods to predict cardiovascular disease risk and/or mortality from electrocardiogram (ECG) are described, in accordance with various embodiments. In several embodiments, ECG waveform data is acquired and one or more ECG waveforms is entered into trained computational model that has been trained to predict future cardiovascular disease events, including mortality. In many embodiments, a prediction using ECG waveform data and a computational model is combined with a prediction from pooled cohort equations (PCE), which can yield an augmented ASCVD prediction that better stratifies a patient's risk. In several embodiments, a clinical intervention, clinical surveillance, and/or a treatment is performed based on a cardiovascular disease risk and/or mortality prediction. In many embodiments, a prediction is utilized to determine whether to treat an individual with a cholesterol reducing medication (e.g., a statin).

The electrocardiogram (ECG) is the most frequently performed cardiovascular diagnostic test, but it is unclear how much information resting ECGs contain about long term cardiovascular risk. Given its low cost and near-ubiquity, the ECG is a good candidate for risk scoring. Unfortunately, there has been limited success in using the ECG to assess cardiovascular risk in the general population (see, e.g., US Preventive Services Task Force et al., JAMA 319, 2308-2314 (2018)). Convolutional Neural Networks (CNNs) trained on large datasets can learn clinically relevant patterns in raw ECG waveforms, often matching or surpassing cardiologist performance on tasks ranging from standard interpretation to diagnosis of near-term diseases like cardiac contractile dysfunction, hypertrophic cardiomyopathy versus hypertension, and atrial fibrillation in patients in sinus rhythm. Furthermore, CNNs are able to predict short-term all-cause and post-operative mortality with high accuracy based on ECG. However, predicting long-term cardiovascular mortality has been challenging despite potentially major implications for clinical intervention such as decisions about statin use.

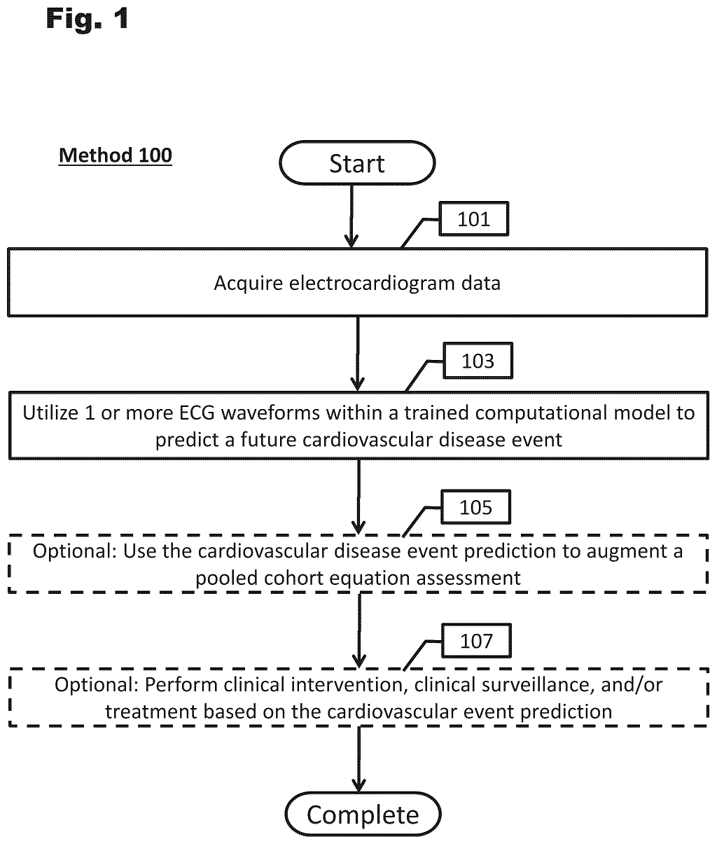

Several embodiments are directed to utilizing a trained computational model to analyze one or more ECG waveforms to predict cardiovascular disease and/or mortality. Provided in FIG. 1 is an exemplary method to predict a future cardiovascular event utilizing one or more ECG waveforms and a trained computational model.

Process 100 begins with acquiring (101) electrocardiogram data. Any methodology to generate ECG waveforms can be utilized. Generally, one or more leads are calculated using one or more electrodes placed on an individual's skin near or around the individual's heart and/or extremities. In some embodiments, 12 leads are collected from ten electrodes (e.g., 12-lead ECG). In some embodiments, ECG data is generated in accordance with a medical standard, such as set by the American Heart Association (AHA), the International Electrotechnical Commission (IEC), the Society for Cardiological Science and Technology (SCST), or any other recognized association.

One or more waveforms generated from the one or more leads is utilized (103) within a trained computational model to predict a future cardiovascular disease event. Future cardiovascular disease events that can be predicted include (but are not limited to) development of atherosclerotic cardiovascular disease (ASCVD), infarction, heart failure, non-lethal heart attack, lethal heart attack, stroke, sudden cardiac death, and any combination of cardiovascular disease events.

In several embodiments, the computational model is trained on a collection of ECG waveform data from a cohort of individuals having cardiovascular health records. Utilizing health records that include the timeline of cardiovascular events after collection of ECG data, a computational model can be trained to recognize waveform data that signifies the cardiovascular events and timeframe in which they are likely to occur. Accordingly, a computational model can be utilized to predict likelihood of a future cardiovascular event within a period of time.

In some embodiments, a computational model predicts the likelihood of a cardiovascular event to occur within one year. In some embodiments, a computational model predicts the likelihood of a cardiovascular event to occur within two years. In some embodiments, a computational model predicts the likelihood of a cardiovascular event to occur within three years. In some embodiments, a computational model predicts the likelihood of a cardiovascular event to occur within four years. In some embodiments, a computational model predicts the likelihood of a cardiovascular event to occur within five years. In some embodiments, a computational model predicts the likelihood of a cardiovascular event to occur within six years. In some embodiments, a computational model predicts the likelihood of a cardiovascular event to occur within seven years. In some embodiments, a computational model predicts the likelihood of a cardiovascular event to occur within eight years. In some embodiments, a computational model predicts the likelihood of a cardiovascular event to occur within nine years. In some embodiments, a computational model predicts the likelihood of a cardiovascular event to occur within ten years.

In some embodiments, a computational model predicts the timeframe in which a cardiovascular will occur. For example, with a certain percent of likelihood, a computational model can predict when a cardiovascular event will occur.

In some embodiments, the computational model is able to predict a cardiovascular event that will occur more than one year into the future. In some embodiments, the computational model is able to predict a cardiovascular event that will occur more than two years into the future. In some embodiments, the computational model is able to predict a cardiovascular event that will occur more than three years into the future. In some embodiments, the computational model is able to predict a cardiovascular event that will occur more than four years into the future. In some embodiments, the computational model is able to predict a cardiovascular event that will occur more than five years into the future. In some embodiments, the computational model is able to predict a cardiovascular event that will occur more than six years into the future. In some embodiments, the computational model is able to predict a cardiovascular event that will occur more than seven years into the future. In some embodiments, the computational model is able to predict a cardiovascular event that will occur more than eight years into the future. In some embodiments, the computational model is able to predict a cardiovascular event that will occur more than nine years into the future. In some embodiments, the computational model is able to predict a cardiovascular event that will occur more than ten years into the future.

Any computational model capable of interpreting ECG waveforms can be utilized, such as those utilized to analyze image data. Computational models that can be utilized include (but are not limited to) deep neural networks (DNN), convolutional neural networks (CNN), recurrent neural networks, long short-term memory (LSTM) networks, kernel ridge regression (KRR), and/or gradient-boosted random forest decision trees. Furthermore, any appropriate model architecture can be utilized that provides an ability to predict a future cardiovascular event. In some embodiments, the computational model is trained on one or more ECG waveform leads. In some embodiments, the computational model is trained on twelve ECG waveform leads.

In many embodiments, the trained computational model is utilized to predict an individual's future risk of a cardiovascular event. Any individual can be assessed, including (but not limited to) a healthy individual, an individual diagnosed with a cardiovascular disorder, an individual with family history of cardiovascular disease, an individual with high blood pressure, an individual that is overweight, and an individual that is obese. Furthermore, an ECG can be performed in relation to a cardiac event or during routine examination. An example of a computational model is detailed in the attached manuscript that describes the Stanford Estimator of Electrocardiogram Risk (SEER) model, which is a CNN-based risk score to predict long-term risk of cardiovascular-related mortality and other disease from a single 12-lead ECG.

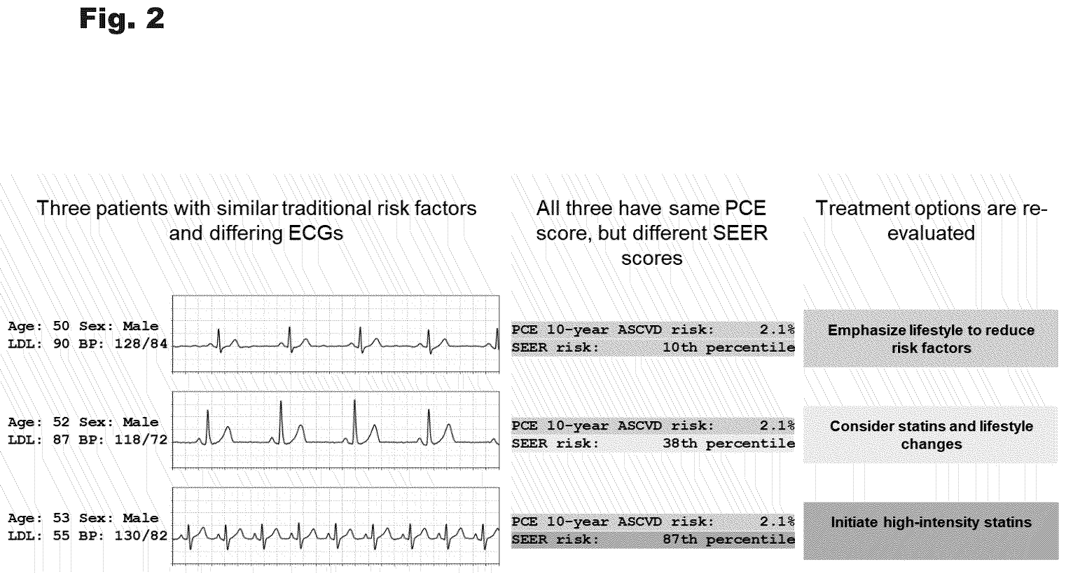

Provided in FIG. 2 is an exemplary assessment of three patients with similar traditional risk factors but have differing ECGs. Using the pooled cohort equation (PCE), which is generally considered the current best estimate of ASCVD risk assessment, the three individuals are predicted to have low ASCVD risk (each predicted to have a 2.1% chance for an ASCVD event within ten years). Using the SEER computational model, which predicts risk of ASCVD in 5 years based on ECG waveforms, the three patients were each predicted to have very different risk assessments and stratified into different risk categories. Accordingly, a computational model that utilizes ECG waveforms can better stratify patients than the current best estimate of ASCVD risk. This stratification can better inform a practitioner of the patient's risks and provide more appropriate care.

Method 100 can optionally use (105) the predicted cardiovascular disease event to augment a PCE assessment. The PCE estimator is based on a combination of established cardiovascular risk factors including age, sex, race, smoking status, systolic blood pressure, hypertension treatment status, diabetes status, and high-density lipoprotein (HDL) cholesterol levels. Risk estimates stratify patients to guide recommendations for preventative therapies, including (but not limited to) lifestyle modification, statin medication, and antihypertension medication.

Risk is stratified into low risk (0 to 7.5% chance of an ASCVD event), moderate risk (7.5 to 20% chance of an ASCVD risk), and high risk (20% or higher). Generally, patients with low risk can continue routine health monitoring and do not need a clinical intervention or treatment. Patients with moderate risk are typically further examined to assess potential ASCVD risks, and may be prescribed a treatment regimen. Patients within the moderate risk group can be difficult to assess as it is unclear whether treatment is necessary. To help better stratify patients within the moderate group, coronary artery calcium (CAC) can be measured, which can sometimes help determine whether to prescribe a statin medication. Patients with high risk are typically prescribed a statin medication and closely monitored by a practitioner. Further assessments are typically performed to determine the amount atherosclerosis is present within the cardiovascular system, whether hypertension or diabetes is present, and whether other treatments are to be performed, including (but not limited to) medicinal treatment of hypertension, medicinal treatment of diabetes, and surgical intervention (e.g., bariatric surgery). As shown in FIG. 2, however, PCE can incorrectly categorize a patient's risk.

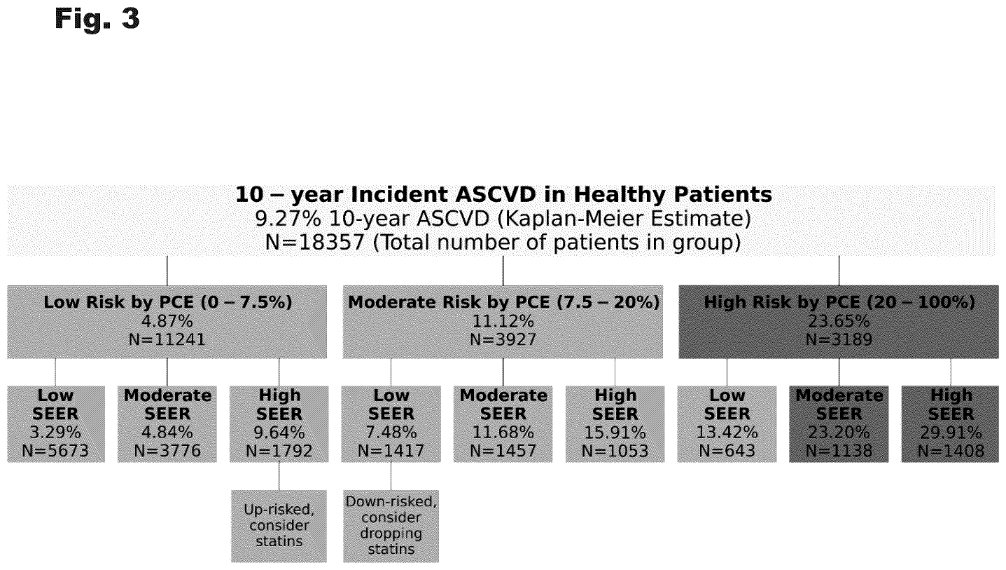

Provided in FIG. 3 are the results of risk stratification of ACSVD events of 18,357 healthy patients within ten years as determined PCE. Most patients (11,241) were categorized into low risk, while 3927 patients were categorized as having moderate risk, and 3189 patients were categorized as having high risk. Using ECG waveforms and the SEER model, the ASCVD risk was further stratified for the patients within each category. For instance, about 16% of the patients determined to have low risk via PCE were determined to have high risk via the SEER model. By combining the two risk scores, many patients having low risk via PCE only were further stratified within a combined category of moderate risk or a high risk of an ASCVD event and thus should be monitored and treated accordingly. Similarly, patients having moderate risk via PCE, were further stratified within a combined category of low risk, moderate risk, or high risk of an ASCVD event and thus should be monitored and treated accordingly.

Method 100 optionally performs (107) a clinical intervention, clinical surveillance, and/or treatment based on the cardiovascular event prediction based on the future cardiovascular disease event prediction. In some embodiments, the future cardiovascular disease event prediction is combined with a PCE risk estimation to determine whether to perform a clinical intervention, clinical surveillance, and/or treatment.

Clinical interventions include clinical procedures and treatments. Clinical procedures include (but are not limited to) blood tests, genetic tests, medical imaging, physical exams, and other cardiovascular health assessments. Clinical surveillance includes continued monitoring by a practitioner, which can be performed by any practical means, including (but not limited to) practitioner visits and routine clinical procedures. Treatments include (but are not limited to) cholesterol reduction medicine, antihypertension medicine, diabetes medicine, surgical procedures, supplements, and lifestyle alterations. Cholesterol reduction medicine includes (but is not limited to) statins, cholesterol absorption inhibitors, PCSK9 inhibitors, citrate lyase inhibitors, and bile acid sequestrants. Antihypertension medicine includes (but is not limited to) diuretics, angiotensin-converting enzyme (ACE) inhibitors, angiotensin II receptor blockers (ARBs), and calcium channel blockers. Diabetes medicine includes (but is not limited to) metformin, sulfonylureas, glinides, thiazolidinediones, DPP-4 inhibitors, GLP-1 receptor agonists, and SGLT2 inhibitors. Surgical procedures include (but is not limited to) bariatric surgery, coronary artery bypass grafting, heart valve repair or replacement, and insertion of a pacemaker or an implantable cardioverter defibrillator. Supplements include (but are not limited to) fibrates, niacin, omega-3 fatty acids, red yeast rice, magnesium, chromium, vitamin D, B vitamins, coenzyme Q10, garlic, cinnamon, and ginseng. Lifestyle alterations include (but are not limited to) cessation of smoking, reduction of caloric intake, increase if exercise, and reduction of stress.

While specific examples of processes to predict a cardiovascular event are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes to predict a cardiovascular event appropriate to the requirements of a given application can be utilized in accordance with various embodiments of the invention.

### Computational Processing System

A computational processing system to predict future cardiovascular events in accordance with various embodiments of the disclosure typically utilizes a processing system including one or more of a CPU, GPU and/or neural processing engine. In a number of embodiments, ECG waveform data is processed to generate a prediction of a cardiovascular event using a computational processing system. In some embodiments, the computational processing system is housed within a computing device that is in direct association the ECG device. In some embodiments, the computational processing system is housed separately from and receives the acquired ECG waveform data. In certain embodiments, the computational processing system is in communication with the ECG device. In various embodiments, the processing system communicates with the ECG device by any appropriate means (e.g., a wireless connection). In certain embodiments, the computational processing system is implemented as a software application on a computing device such as (but not limited to) mobile phone, a tablet computer, a wearable device (e.g., watch), and/or portable computer. In some embodiments, a wearable device incorporates one or more leads such that it acquires the ECG waveform data and further processes the data to predict a cardiovascular event. It is to be understood that a wearable device is one that is portable and can be utilized in a nonclinical setting, such as (for example) a smart watch, wearable chest straps, and smart clothing.

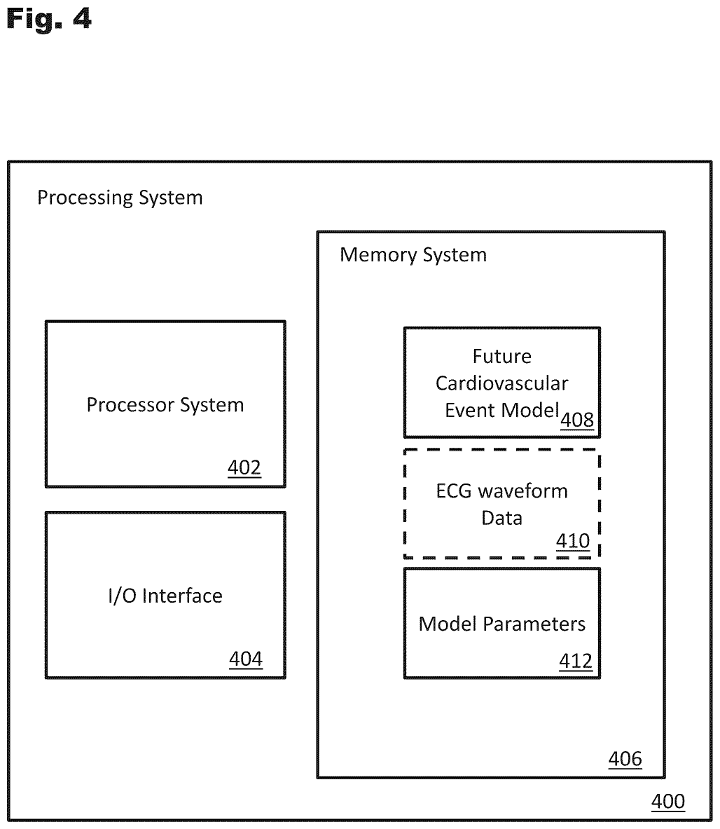

A computational processing system in accordance with various embodiments of the disclosure is illustrated in FIG. 4. The computational processing system 400 includes a processor system 402, an I/O interface 404, and a memory system 406. As can readily be appreciated, the processor system 402, I/O interface 404, and memory system 406 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a future cardiovascular event model application 408. The future cardiovascular event model application can be downloaded and/or stored in non-volatile memory. When executed, the future cardiovascular event model application is capable of configuring the processing system to implement computational processes including (but not limited to) the computational processes described above and/or combinations and/or modified versions of the computational processes described above. In several embodiments, the future cardiovascular event model application 408 utilizes ECG waveform data 410, which can be stored in the memory system, to perform signal processing in order to predict future cardiovascular events. In some embodiments, the memory includes an application for performing an electrocardiogram, including collecting electrical signals of an individual and generating ECG waveform data. In certain embodiments, the future cardiovascular event model application 408 utilizes model parameters 412 stored in memory to process acquired ECG waveform data. In several embodiments, the ECG waveform data 210 is temporarily stored in the memory system during processing and/or saved for use in establishing model parameters.

While specific computational processing systems are described above with reference to FIG. 4, it should be readily appreciated that computational processes and/or other processes utilized in the provision of future cardiovascular event prediction in accordance with various embodiments of the disclosure can be implemented on any of a variety of processing devices including combinations of processing devices. Accordingly, computational devices in accordance with embodiments of the disclosure should be understood as not limited to specific ECG systems, computational processing systems, and/or cardiovascular event risk models. Computational devices can be implemented using any of the combinations of systems described herein and/or modified versions of the systems described herein to perform the processes, combinations of processes, and/or modified versions of the processes described herein.

### Examples

The embodiments of the disclosure will be better understood with the various examples provided herein. Described below are examples comparing standard practices of estimating cardiovascular mortality and ASCVD risk with methods as described herein.

Using a dataset of 910,966 resting 12-lead ECGs collected at Stanford University Medical Center, the Stanford Estimator of Electrocardiogram Risk (SEER) was developed. SEER predicts five-year cardiovascular mortality with an area under the receiver operator characteristic curve (AUC) of 0.83 in a held-out test set at Stanford, and with AUCs of 0.79 and 0.83 when independently evaluated at Cedars-Sinai Medical Center and Columbia University Irving Medical Center respectively. SEER predicts 5-year atheroscleroitc disease (ASCVD) with an AUC of 0.67 and is close in performance to the Pooled Cohort Equations for ASCVD Risk while being only modestly correlated. When used in conjunction with the Pooled Cohort Equations, SEER accurately reclassified 16% of patients from low to moderate risk, uncovering a group with an actual average 9.64% 10-year ASCVD risk who would not have otherwise been indicated for statin therapy. SEER can also predict several other cardiovascular conditions such as heart failure and atrial fibrillation. Its performance is consistent across various demographic groups. Using only lead I of the ECG, in a setup similar to that of a smartwatch ECG, it predicts five-year cardiovascular mortality with an AUC of 0.79. SEER, used alongside the Pooled Cohort Equations and other risk tools, can significantly improve cardiovascular risk stratification and aid in medical decision making.

### Study Population

SEER was primarily trained and evaluated using a dataset of 910,966 resting ECGs from 307,557 patients at Stanford University Medical Center. A set of 312,422 ECGs with five years of followup was used to train SEER to predict five-year cardiovascular mortality. SEER was first evaluated using cross-validation on the Stanford cross-validation set, a dataset of 244,839 ECGs. Also provided are results on the Stanford PCE comparison set, a subset of the Stanford cross-validation set with 18,357 ECGs associated with clinical data to fairly compare SEER to the PCE. In addition, results were gathered on three held-out test sets, the Stanford, Cedars-Sinai Medical Center (Cedars-Sinai), and Columbia University Irving Medical Center (Columbia) test sets of 31,899, 46,795, and 458,455 ECGs.

### SEER Accurately Predicts Cardiovascular Mortality and ASCVD Events

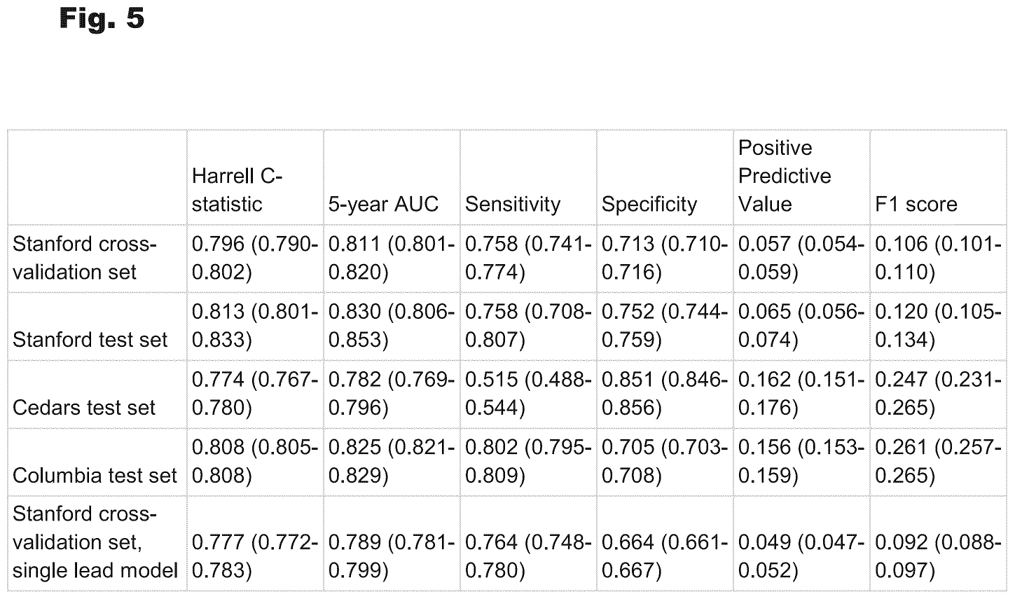

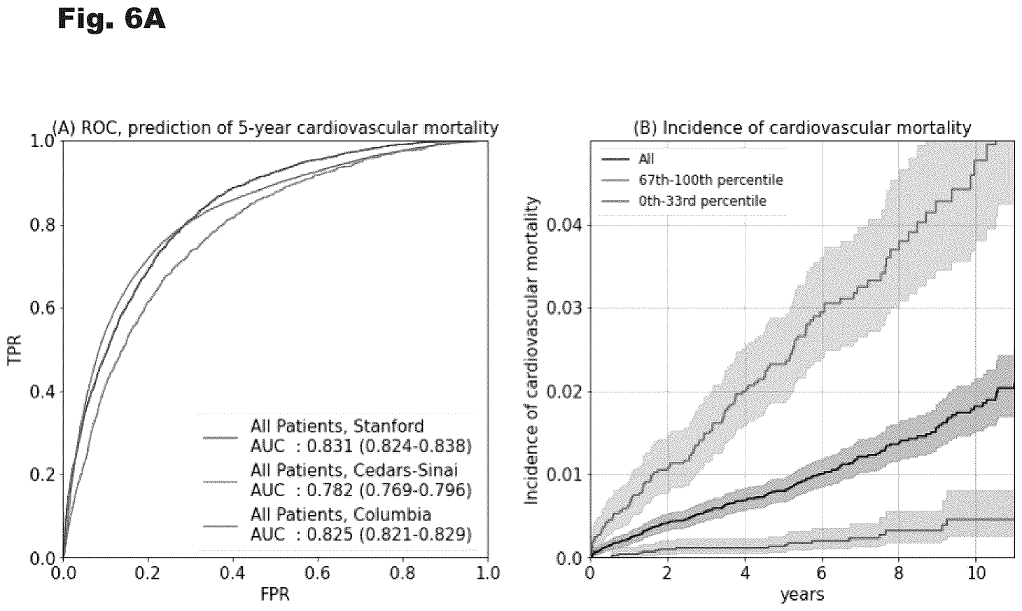

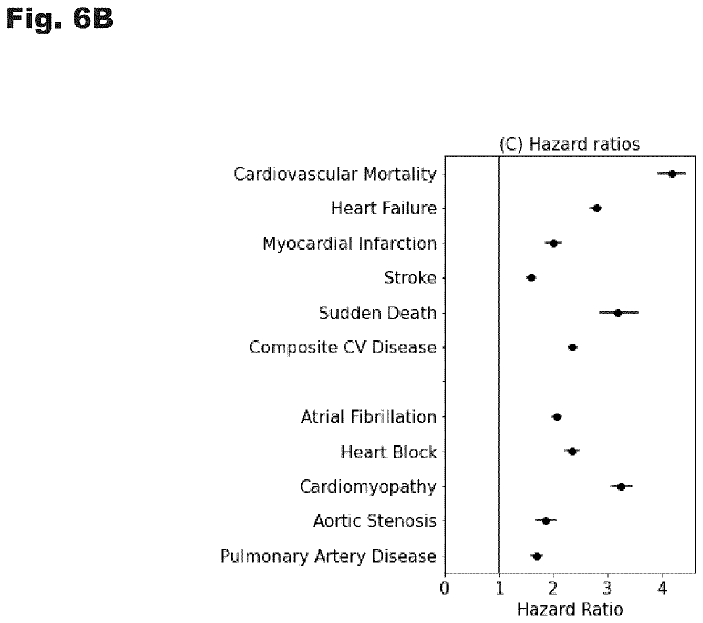

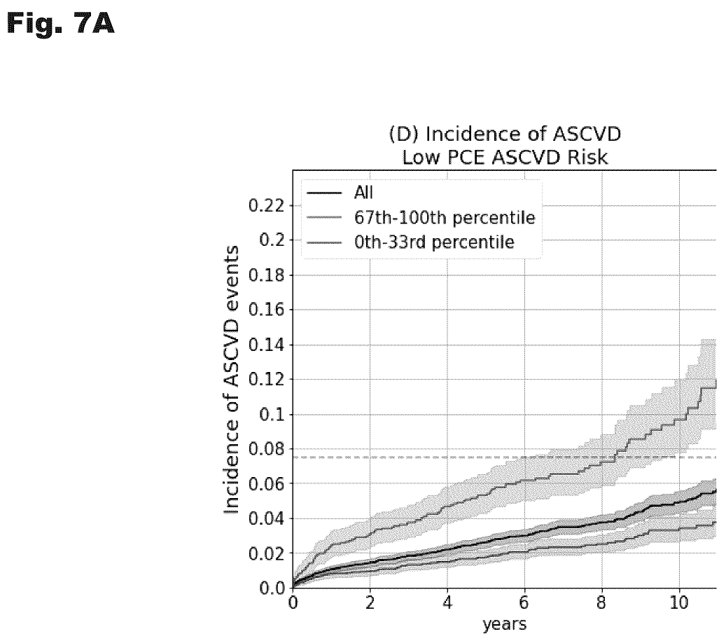

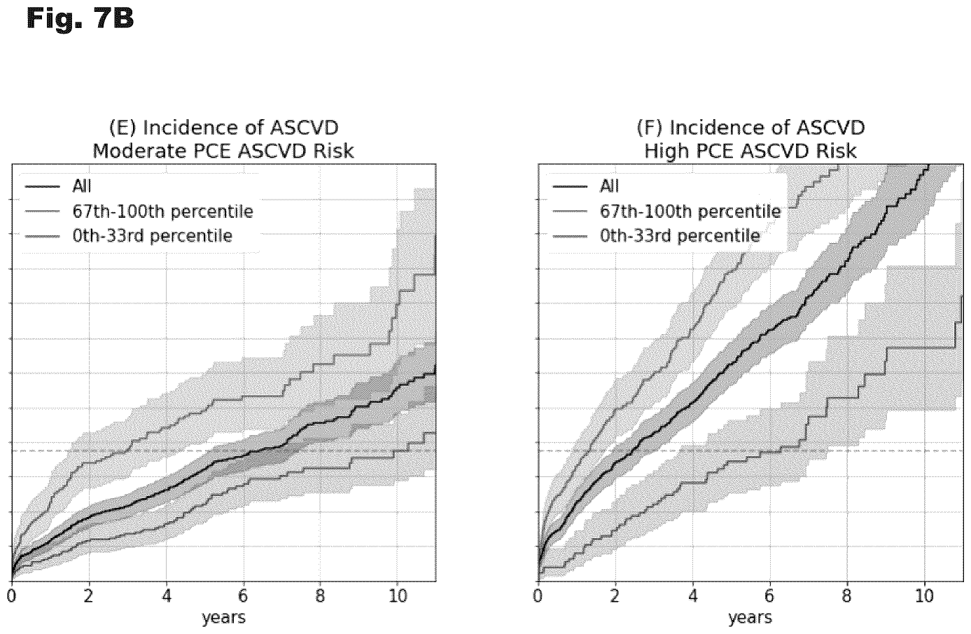

SEER's performance was investigated in predicting cardiovascular mortality based on a single 12-lead ECG in the Stanford cross-validation set (FIG. 5). Cardiovascular mortality was defined as a mortality falling within thirty days of a myocardial infarction, ischemic stroke, intracranial hemorrhage, sudden cardiac death, or hospitalization for heart failure. Among patients in that set with five years of followup or a cardiovascular mortality within five years, SEER predicted five-year cardiovascular mortality with area under the receiver operator characteristic curve (AUC) of 0.81 (95% CI: 0.80-0.82) (FIG. 7A). While SEER was trained on the binary five-year prediction task, it was similarly accurate in predicting relative survival times, achieving a Harrell's C-statistic of 0.80 (0.79-0.80) (see F. Harrell, et al., Stat. Med. 15, 361-387 (1996), the disclosure of which is incorporated herein by reference). Using the top tertile as a cutoff, SEER balanced sensitivity and specificity well at 0.76 (0.74-0.78) and 0.71 (0.71-0.72) respectively. It achieved a positive predictive value of 0.06 (0.05-0.06), i.e. 6% of patients in the top third of SEER risk suffered a cardiovascular mortality within 5 years. Being in the top third of SEER indicated a 4.2 (4.0-4.4) fold age and sex-adjusted increase in hazard of cardiovascular mortality. SEER stratified risk across all time scales between a few days and over ten years (FIG. 6A).

Patients in the top third of the SEER score were at higher risk for developing a range of incident cardiovascular diseases (FIG. 6B). In the Stanford cross-validation set, age and sex-adjusted hazard ratios were 2.8 (2.7-2.9) for heart failure, 2.0 (1.9-2.1) for myocardial infarction, 1.6 (1.5-1.7) for stroke, and 3.2 (2.9-3.5) for sudden cardiac death. For a composite of those four events, the adjusted hazard ratio was 2.4 (2.3-2.4). The trend was similar for other cardiovascular conditions including atrial fibrillation (2.1 (2.0-2.1)), heart block (2.3 (2.2-2.5)), cardiomyopathy (3.3 (3.1-3.4)), aortic stenosis (1.9 (1.7-2.0)), and peripheral vascular disease (1.7 (1.6-1.8)).

SEER was additionally evaluated on the Stanford, Cedars-Sinai, and Columbia test sets. The Cedars-Sinai and Columbia test sets consisted of ECGs from General Electric ECG machines, while all Stanford sets including the training set consisted of ECGs from Philips machines. Among the three test sets, it performed similarly, with AUCs of between 0.78 and 0.83 (CIs extending between 0.76 and 0.85) and Harrell's C-statistics between 0.77 and 0.81 (0.76 to 0.82) (FIG. 6A). Positive predictive values at Cedars-Sinai and Columbia were higher, likely due to higher rates of disease overall.

### SEER Complements the PCE Risk Score

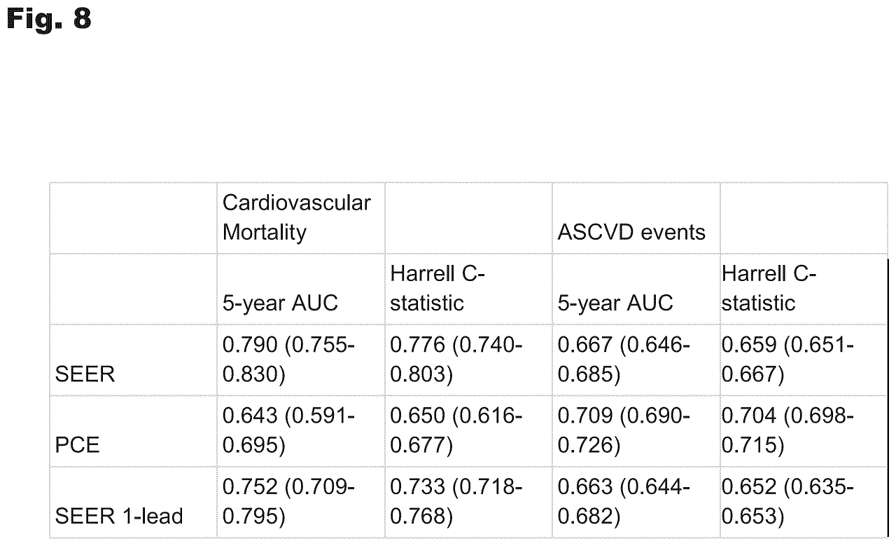

SEER's performance was assessed on the Stanford PCE comparison set, comprising outpatients who had never suffered a prior cardiovascular disease event, were non-diabetic, had an LDL cholesterol measurement below 190 mg/dL, and had a blood pressure measurement within the year prior to the ECG. These criteria were selected to closely represent the set of patients eligible for risk screening for long-term cardiovascular disease, and to allow us to compare SEER to the PCE. In this set, SEER predicted cardiovascular mortality with a five-year AUC of 0.79 (0.76-0.83), and a Harrell C-statistic of 0.78 (0.73-0.78) based on a single 12-lead ECG (FIG. 8). Within the same set, the PCE achieved a five-year AUC of 0.64 (0.59-0.70) and a Harrell C-statistic of 0.65 (0.61-0.67).

SEER's ability to predict incident hard atherosclerotic cardiovascular disease events (ASCVD) was additionally evaluated in the Stanford PCE comparison set, using the standard composite endpoint of lethal and non-lethal myocardial infarction, stroke, and sudden cardiac death. SEER achieved a 5-year AUC of 0.67 (0.65-0.69) and Harrell C-statistic of 0.66 (0.65-0.68) in predicting hard ASCVD, while the PCE performed slightly better, with a 5-year AUC of 0.71 (0.69-0.73) and Harrell C-statistic of 0.70 (0.69-0.71). SEER and the PCE score were only modestly correlated with a Pearson correlation of 0.218 (P<10⁻¹⁹⁵), and are based on different data modalities.

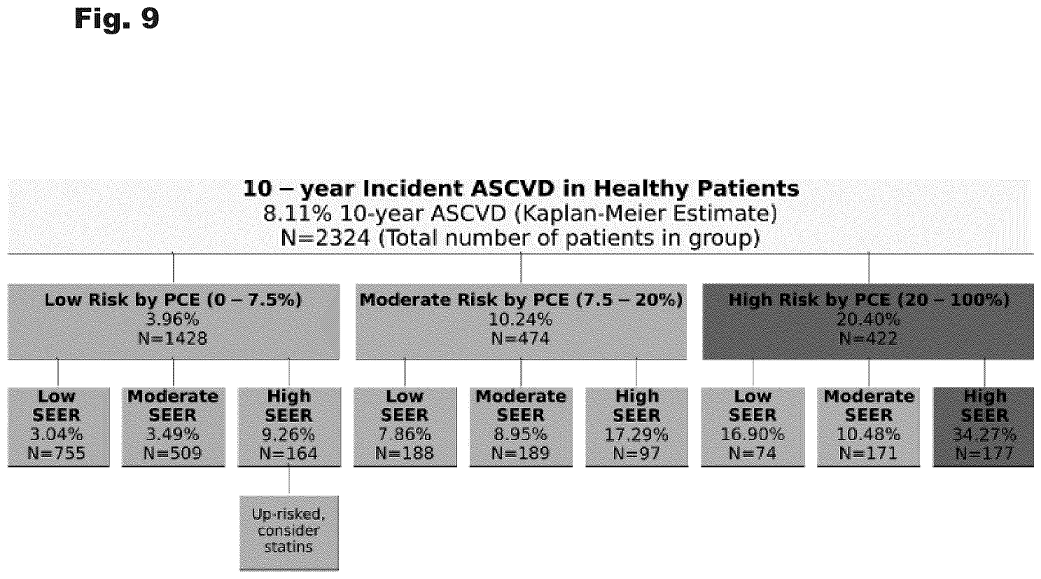

To understand how SEER might fit into current clinical practice, it was next explored how it classified patients versus the PCE score in the Stanford PCE comparison set (Fig. e). Various groups of risk were considered, including low, moderate, and high risk as determined by the PCE risk score, and it was examined how SEER would have classified them. SEER was used to separate patients into three tertiles of risk based on cutoffs at the bottom and top thirds of the cross-validation set. The 11,241 patients categorized as low-risk by the PCE risk score (with a PCE-estimated 10-year ASCVD rate below 7.5%) had an actual 10-year ASCVD rate of 4.87% (Kaplan-Meier estimate; 95% CI 4.30%-5.53%) and a 10-year cardiovascular mortality rate of 1.05% (0.79%-1.38%) (FIG. 7A). Within that group, the 1,792 patients with a SEER score in the top tertile had a 10-year ASCVD rate of 9.64% (7.77%-11.93%), above the 7.5% cutoff for recommending statins, and a significantly higher cardiovascular mortality rate of 3.52% (2.44%-5.08%). SEER therefore reclassified 16% of patients classified as low risk by PCE into a moderate risk category, identifying additional patients who may benefit from statin therapy. SEER is also able to reclassify patients with moderate 10-year ASCVD risk. Among the 3,927 patients with moderate ASCVD risk (7.5-20%) according to the PCE score, the 1,417 with a low SEER risk score had a 10-year ASCVD rate of 7.48% (5.78%-9.64%), while the 1,053 with high SEER risk had a rate of 15.91% (12.43%-20.24%) (FIG. 7B). Over 35% of patients were reclassified as being slightly below the 7.5% statin cutoff, and over 25% had significantly higher risk than the moderate PCE risk group overall. Those patients were similarly stratified with respect to cardiovascular mortality risk. SEER also stratified patients with high PCE scores, with patients with low SEER risk experiencing a 13.42% (9.84%-18.17%) 10-year ASCVD rate versus 29.91% (25.60%-34.76%) for patients with high SEER risk (FIG. 7B). Risk of cardiovascular mortality followed a similar pattern of up and down-risking (FIG. 9).

### SEER Performs Well Across Diverse Populations

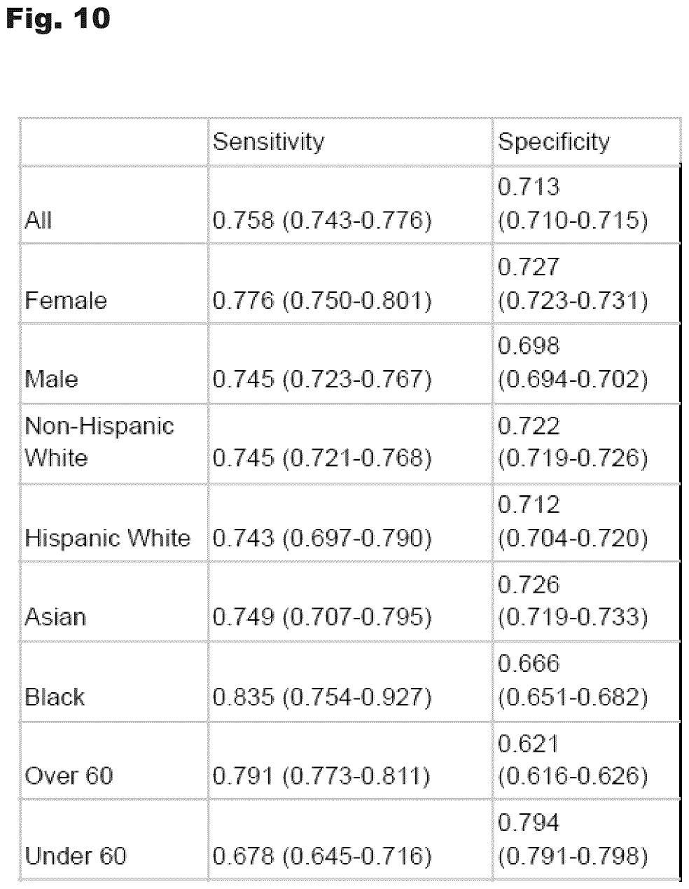

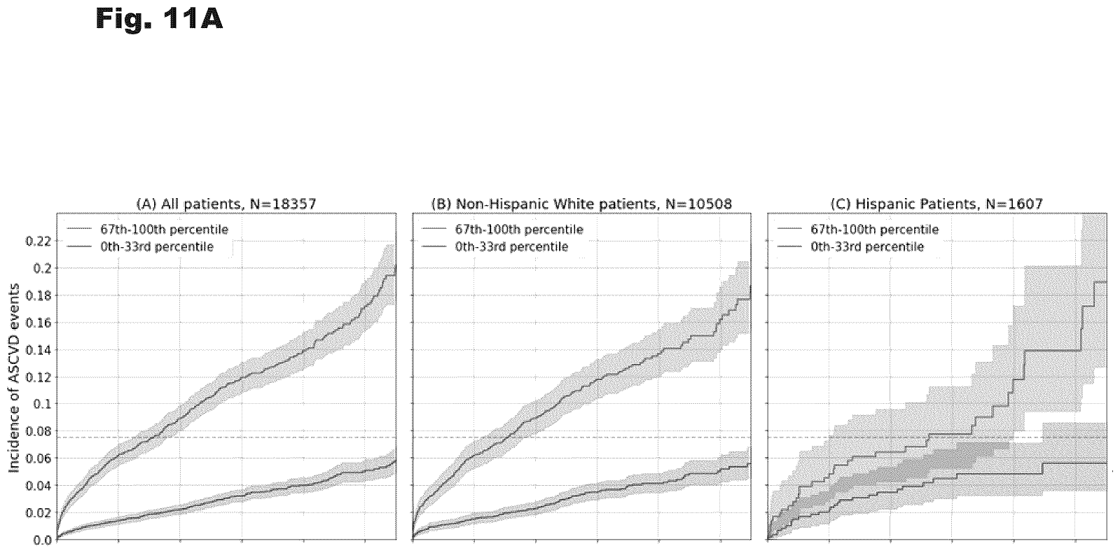

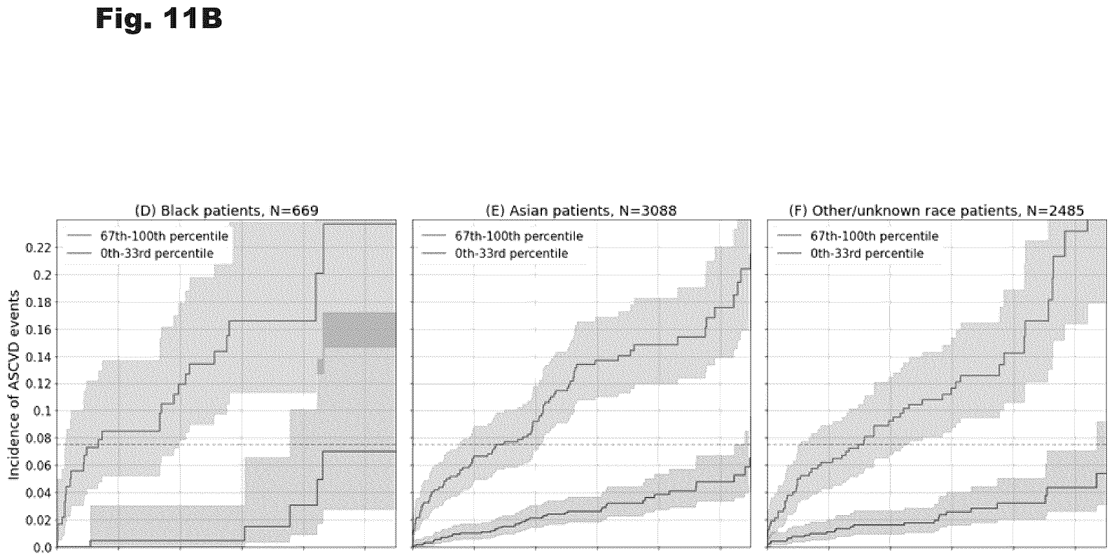

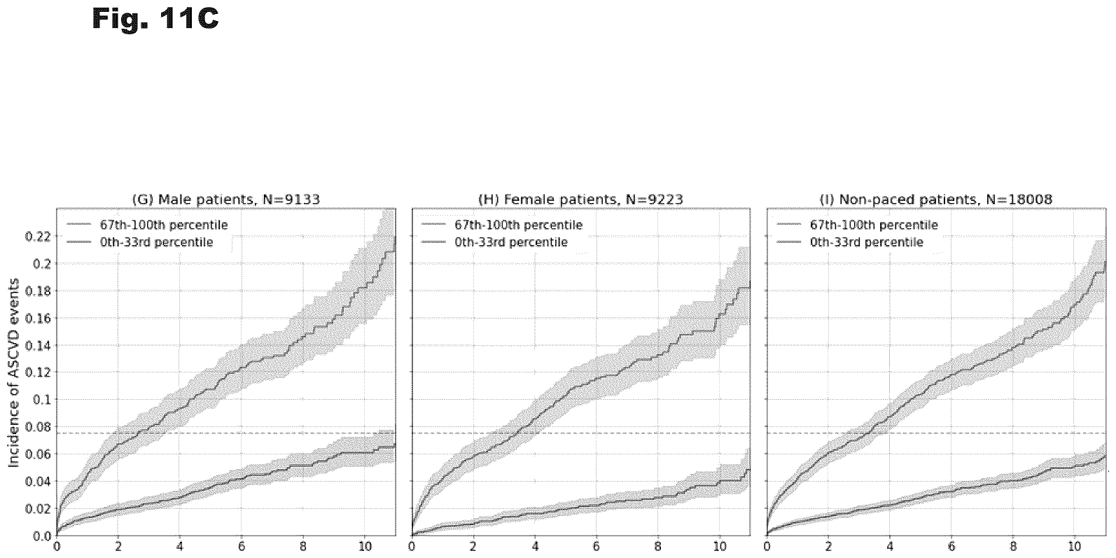

To understand potential biases in SEER, additional validation was performed on a range of demographic subgroups in the Stanford cross-validation set (FIG. 10). To mirror expected clinical use, a cutoff was set for positive prediction at the top third of patients and compared sensitivity and specificity, rather than comparing using the AUC and Harrell C-statistic which only capture within-group discrimination. For most race, ethnicity and sex sub-groups, performance was not significantly different from the entire population. For patients under 60, specificity was significantly higher and sensitivity was significantly lower, and for patients over 60 and black patients, sensitivity was significantly higher and specificity was significantly lower. Even these significant effects were relatively small in size. Survival curves for ASCVD for various demographic groups are shown in FIGS. 11A to 11C, demonstrating robust differentiation across groups.

SEER-Based Risk Correlates with High-Risk ECG and Clinical Features

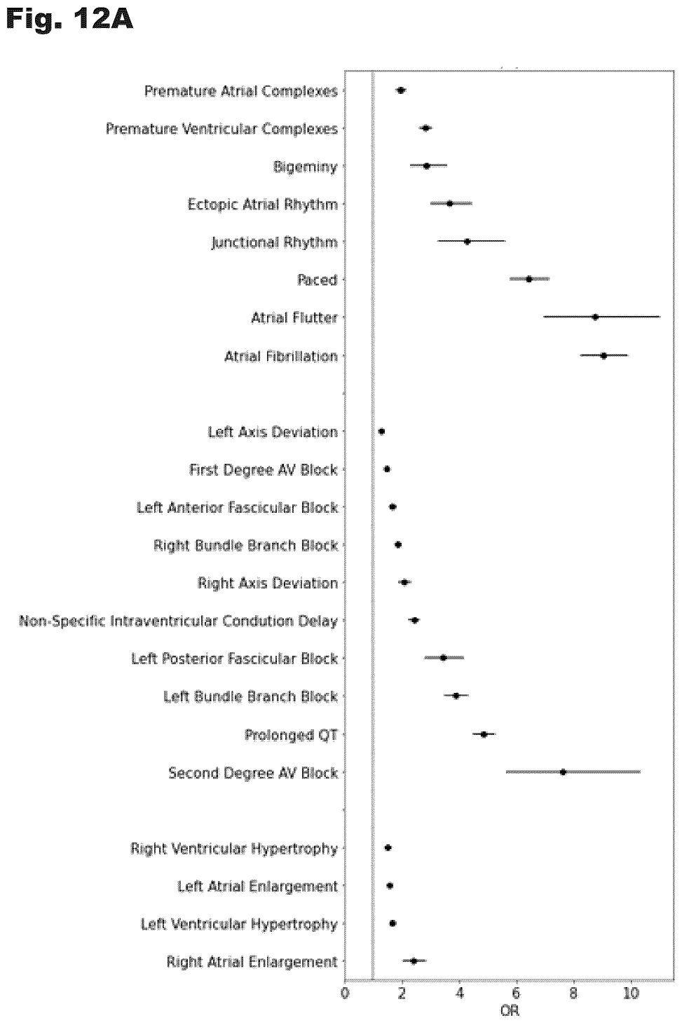

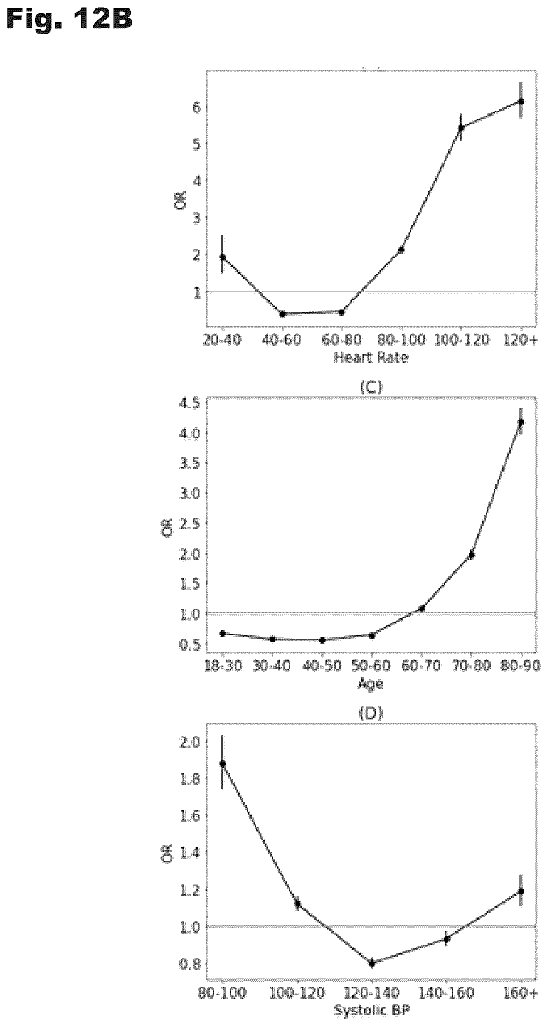

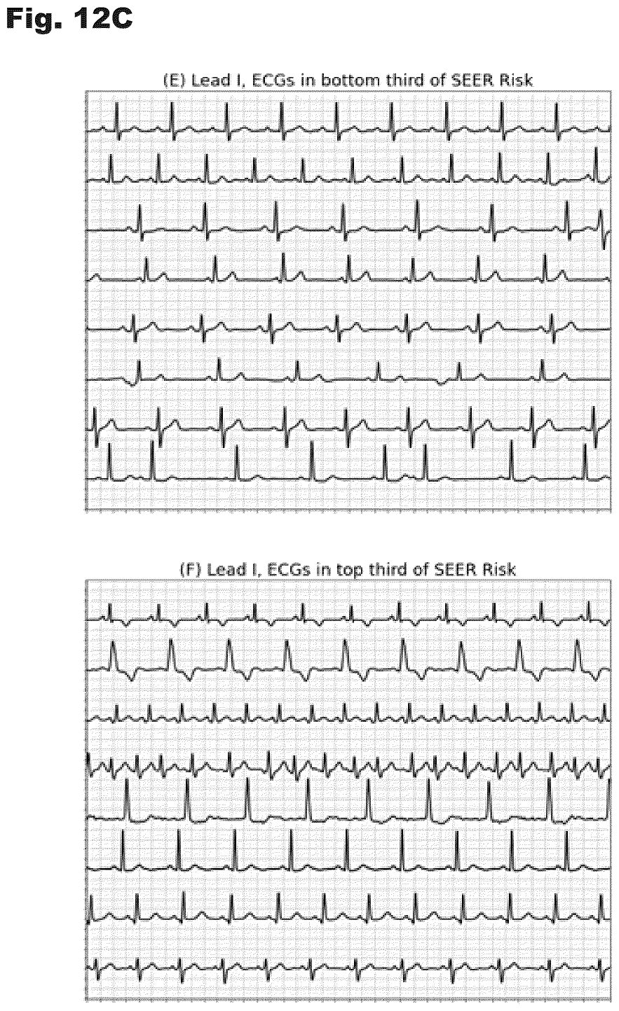

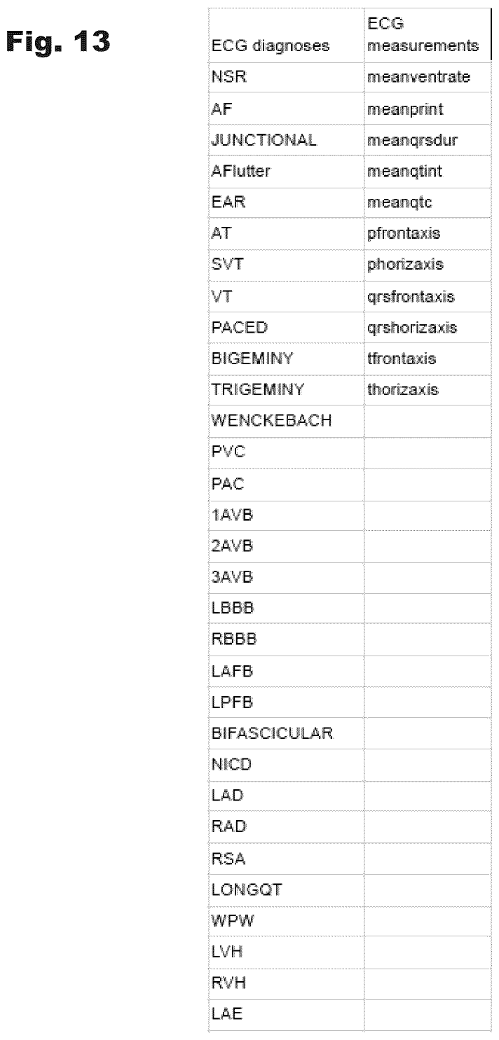

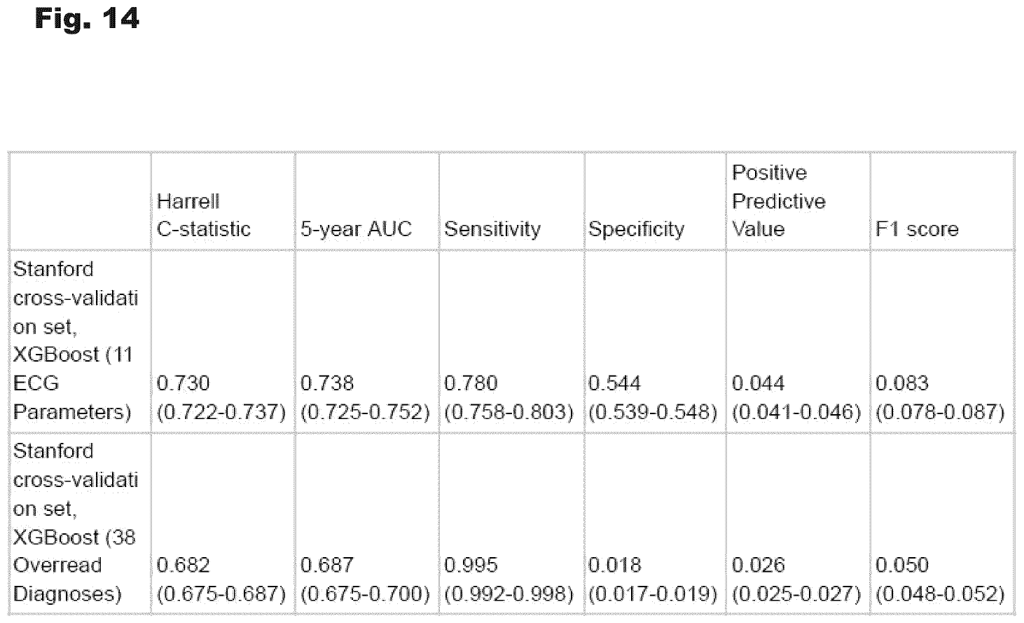

Understanding how well-known ECG risk markers affect the SEER score is a challenge, as the model does not take them directly as inputs (for example, the model does not receive a binary “atrial fibrillation” label, but rather a waveform from which it might extract features related to heart rate variance). To address this issue, odds ratios were utilized to interpret neural net outputs in a novel way. For each of 16 features parsed from the ECG physician overread, the age and sex-adjusted odds ratio of falling in the top third of SEER scores were calculated given each clinician overread-based diagnosis in the Stanford cross-validation set (FIG. 12A). All odds ratios were above 1. The features with lowest odds ratios were left axis deviation, first-degree AV block, and right ventricular hypertrophy, with odds ratios of 1.3 (1.2-1.3), 1.5 (1.4-1.6), and 1.50 (1.4-1.6). The highest were second-degree AV block, atrial flutter, and atrial fibrillation, with odds ratios of 7.6 (5.7-10.3), 8.8 (7.0-11.0), and 9.0 (8.3-9.9). To understand the degree to which SEER relies on standard ECG markers, a random forest model was trained on 38 standard ECG markers (listed in FIG. 13), which achieved an AUC of 0.69 (0.68-0.70) in predicting five-year cardiovascular mortality (FIG. 14; versus SEER's AUC of 0.81).

Increased and severely decreased heart rate both were associated with higher SEER risk, (FIG. 12B), reflecting the elevated risk associated with bradycardia and tachycardia. SEER also correlated with some continuous PCE risk factors. It closely reflected the true risk of elevated age (FIG. 12B). Still, it was not completely driven by age, with a Pearson correlation of 0.18 (P<10⁻¹³⁹). Increased risk among 20-40 year olds was likely due to ascertainment bias of only sicker young patients receiving ECGs. SEER also captured the risk of low blood pressure (FIG. 12B) associated with heart failure, but only predicted a slight increase in risk for elevated blood pressure.

FIG. 12C depicts 8 randomly selected low-risk and high-risk ECG examples. Qualitatively, the ECGs in the top panel appear mostly normal: all ECGs are sinus rhythm, and the only abnormalities noted are premature complexes and 1st degree AV Block. Conversely, the ECGs in lower panel show a range of arrhythmias and morphologic abnormalities.

### SEER Performs Well Using a Single ECG Lead and Outperforms More Limited Data

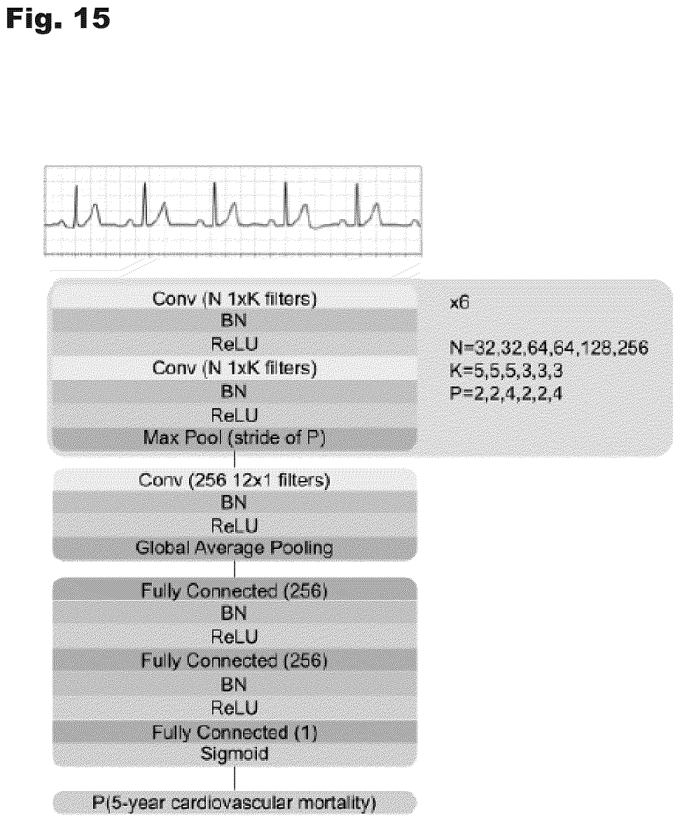

The main SEER model makes predictions based on 12-lead ECG waveforms. To understand which features are important for prediction, we additionally trained and evaluated models on more limited input data. Using only lead I of each ECG, SEER was still able to predict five-year cardiovascular mortality in all patients with an AUC of 0.79 (0.78-0.80) in the Stanford cross-validation set, a less than 0.02 drop in AUC from the 12-lead model (FIG. 5). Provided in FIG. 15 is a reproduction of the 12-lead model except using the single-lead model instead. A random forest model based on the 11 common ECG parameters generated by the Philips Tracemaster software achieved an AUC of 0.72 (0.67-0.77) and a Harrell's C-statistic of 0.71 (0.67-0.72; FIG. 14). This result suggests that SEER makes substantial use of features other than the ones used in standard ECG algorithms.

### Study Populations and Data Sources

SEER was trained, developed, and primarily evaluated using a dataset of resting ECGs from Stanford University Medical Center (Stanford) consisting of all non-low-quality ECGs from patients above the age of 18 taken during the course of clinical care between March 2008 and May 2018. In total we extracted 910,966 ECGs from 307,557 patients from the Phillips TraceMaster system. All ECGs were saved as 10 second signals from all 12 leads of the ECG, sampled at 500 Hz. Band pass and wandering baseline filters were applied to the signals, which were normalized on a per-lead basis, and also downsampled to 250 Hz for performance reasons. Measurements and text overreads were also extracted from TraceMaster, and ECG diagnoses were extracted from text cardiologist overreads using string matching. ECGs were randomly partitioned by patient into the training/cross-validation, validation, and test sets in an 8:1:1 ratio. For training and validation, ECGs that were considered were a cardiovascular mortality (defined below) within five years after the ECG, or more than five years of followup after the ECG (defined in detail below), resulting in 312,422 (39,074) ECGs in the training (validation) set. Model parameters were fit using the training set, and hyperparameters were chosen based on the validation set. All ECGs from each patient were used during model training. All model development, training, and hyperparameter selection was performed using this split.

During model evaluation, the first ECG from each patient was only considered. Once a final model was selected, eight-fold cross-validation was performed on the training set to obtain model predictions on 244,839 ECGs from a set of 244,839 patients who were not part of the validation or test set (not all of whom had five years of followup), to yield the cross-validation set. Cross-validation predictions on each fold were generated based on models trained on all other cross-validation folds and the validation set. Results were also generated on the PCE comparison set, the subset of the cross-validation set consisting of 18,357 non-inpatients who had never suffered a prior cardiovascular disease event, were non-diabetic, and had an LDL cholesterol measurement below 190 mg/dL and any blood pressure measurement within the year prior to the ECG.

To understand how SEER performs on a range of populations, evaluated SEER was additionally on three held out test sets from Stanford, Cedars-Sinai Medical Center (Cedars-Sinai), and Columbia University Irving Medical Center (Columbia). The Stanford test set consists of 31,899 first resting ECGs, from patients not in the Stanford training or validation sets. The Cedars-Sinai test set consists of 46,795 first resting ECGs taken at Cedars-Sinai from the General Electric MUSE system, with mortality and event data from EPIC Clarity. The Columbia test set consists of 458,455 ECGs first resting ECGs taken at Columbia from the General Electric MUSE system, with mortality and event data from their OMOP database. Demographic data for the Cedars-Sinai and Columbia test sets are in supplemental tables 4 and 5.

Followup mortality and disease data were queried from STARR-OMOP, a common data model for accessing Stanford electronic health records, and extended to December of 2020 for model training and February of 2022 for evaluation. A primary outcome of interest was cardiovascular mortality, defined as a mortality in the EHR falling within thirty days of a condition-record of myocardial infarction, ischemic stroke, intracranial hemorrhage, sudden cardiac death, or hospitalization for heart failure. During training ECGs with a cardiovascular mortality within five years of the ECG or five years of followup after the ECG were only considered, defined as a measurement, admission, or mortality more than five years after the ECG. The same definition was used for censoring times in survival analyses. The same OMOP queries were used on Columbia's OMOP database to pull outcomes. Separate queries were written for Cedars-Sinai's EPIC Clarity-based system.

Additional data was queried from STARR-OMOP as selection criteria and for the computation of the pooled cohort equations risk score. Blood pressure and cholesterol measurements were taken within the year prior to the ECG. Smoking, diabetes, and antihypertensive status were determined using any label prior to the ECG, and in the case where there was no prior label were by default set to false. Atherosclerotic cardiovascular disease was defined as the first incidence of myocardial infarction, ischemic stroke, intracranial hemorrhage, or sudden cardiac death in the electronic health record. Atrial fibrillation, heart block, cardiomyopathy, pulmonary artery disease, and aortic stenosis were all defined as the first incidence in the electronic health record.

### Model Development and Training

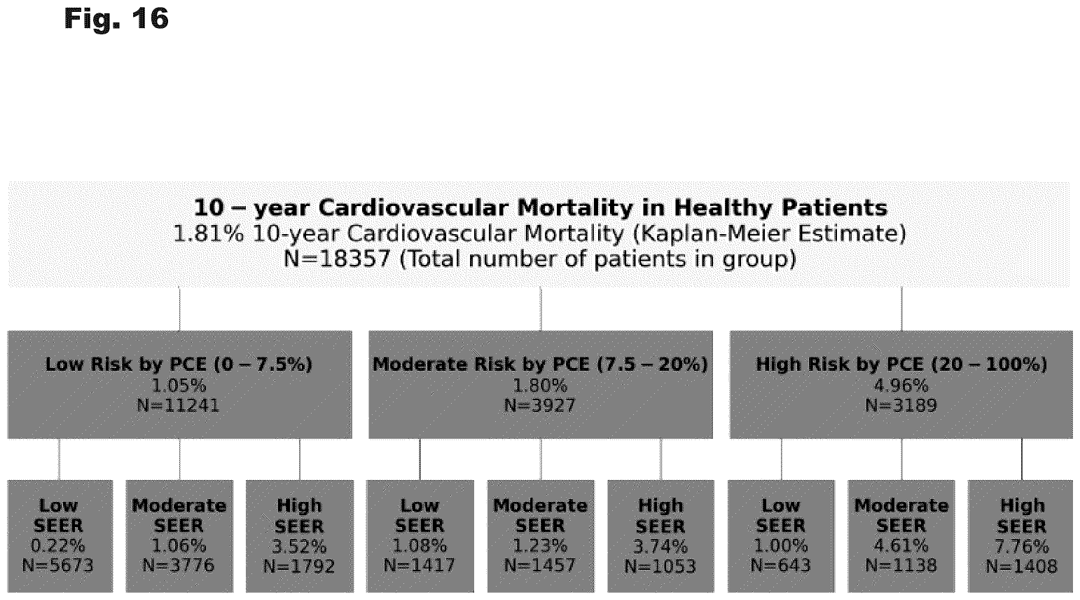

A convolutional neural net was trained to predict five-year cardiovascular mortality among ECGs with either a positive event within five years or a record in the EHR more than five years afterwards. Model development was performed using Python 3.9 and PyTorch 1.11, and models were trained on single Nvidia Titan Xp GPUs using Stanford's Sherlock computing cluster. several convolutional architectures were explored and the one yielding the highest validation accuracy was chosen, as described in FIG. 16. Convolutional architectures are well-suited to ECG data due to the repetition of motifs across time and examples, allowing for convolutional filters to be fit to share information and reduce complexity. The model was chosen and all hyperparameters were tuned by training on the training set and evaluating on the validation set. A batch size of 128, a weight decay hyperparameter of 10⁻⁴, and the ADAM optimizer were used. The learning rate was initialized to 10⁻³ and reduced by a factor of 10 each time the validation loss plateaued for more than five epochs, and training stopped once the learning rate fell to 10⁻⁶. Models were selected based on area under the receiver operator characteristic curve (AUC).

Once a model and hyperparameters were chosen, eight more models were trained using cross-validation on the training and validation sets to generate model predictions on the portions of the training set not used to evaluate the model during training. The results of those eight models and the original model were averaged to make predictions on the test set. All results are based on models trained at Stanford. ECGs from Cedars-Sinai Medical Center and Columbia Medical Center were treated exactly as ECGs at Stanford, downsampled from 500 to 250 Hz, pre-processed using band pass and high pass wandering baseline filters, and normalized per-lead, based on normalization parameters specific to Cedars-Sinai. Both Cedars-Sinai and Columbia use the General Electric MUSE ECG software and General Electric ECG machines.

The continuous model prediction was converted to a categorical risk prediction by taking the two tertiles of the SEER score in the Stanford cross-validation set (i.e. the 33.3 . . . and 66.6 . . . percentiles). All references to bottom and top thirds of model predictions are based on the cutoffs from this group, including validation at other sites and experiments in the Stanford PCE comparison set. These cutoffs are equivalent to 1.1% and 3.9% risk of cardiovascular mortality (which should not be directly compared to 10-year risk of ASCVD).

Single lead ECG models were trained using the same architecture and hyperparameters as 12-lead models, but using only lead I of the ECG and using 1 by 1 convolutions in place of the 1 by 12 convolutions. Random forest models were developed and trained using XGBoost 1.5, using the features in supplementary table 6.

### Statistical Analysis

Models were primarily compared based on the area under the receiver operator characteristic (AUC) and the Harrell's C-statistic. The former is a standard metric used for evaluating stratification in binary classification tasks, while the latter is a similar score for evaluating stratification in survival prediction tasks with censoring. The AUC was computed using the scikit-learn Python package, and 95% confidence intervals were constructed using the bootstrap method with 100 samples. Unless otherwise noted, all binary metrics were computed at a five-year time horizon, comparing all examples with an event within five years versus all examples with no event but other followup data after five years. The c-statistic was computed using the lifelines Python package, and 95% confidence intervals were constructed using the bootstrap method with 100 samples. C-statistics were computed including the entire population. Sensitivity, specificity, and positive predictive values were additionally computed using standard definitions and using the top tertile as the cutoff for positive predictions.

Hazard ratios were computed to measure how predictive SEER is of future outcomes, and odds ratios to measure how current ECG and clinical features affect SEER. Hazard ratios were calculated using the lifelines Python package using Cox proportional hazards models, correcting for age and sex. All hazard ratios indicate the hazard of a patient being in the top third of SEER risk. Kaplan-Meier estimates were computed using the lifelines Python package. All confidence intervals on Kaplan-Meier curves are 95% Kaplan-Meier confidence intervals. The observed event rates in FIG. 3 are Kaplan-Meier estimates, since the high number of censoring events would otherwise bias the event rates to be higher. Odds ratios were calculated using the statsmodels Python package using logistic regression models correcting for age and sex (with the exception of the odds ratios for age, which were only corrected for sex). All odds ratios indicate the odds of a patient being in the top third of SEER risk given the characteristic.

What is claimed is:

1. A computational method for predicting a future cardiovascular event, comprising:
obtaining, using a computational processing system, electrocardiogram data derived from an individual, wherein the electrocardiogram data comprises one or more electrocardiogram waveforms; and
predicting, using the computational processing system and a trained computational model, a risk that the individual will experience a cardiovascular disease event in the future, wherein the trained computational model utilizes the one or more electrocardiogram waveforms to predict a likelihood of the cardiovascular disease event.

2. The method of claim 1, wherein the trained computational model is trained from electrocardiogram data obtained from a cohort of individuals having cardiovascular health records that include a timeline of cardiovascular events after collection of each individual's electrocardiogram data.

3. The method of claim 1, wherein the computational model is a deep neural network (DNN), a convolutional neural network (CNN), a recurrent neural network, a long short-term memory (LSTM) network, a kernel ridge regression, or a gradient-boosted random forest decision tree.

4. The method of claim 1, wherein the trained computational model predicts the risk that the individual will experience a cardiovascular disease event that will occur in more than one year.

5. The method of claim 1, wherein the trained computational model predicts the risk that the individual will experience a cardiovascular disease event that will occur within five years.

6. The method of claim 1, wherein the trained computational model predicts the risk that the individual will experience a cardiovascular disease event that will occur within ten years.

7. The method of claim 1, wherein the cardiovascular event is development of atherosclerotic cardiovascular disease (ASCVD), infarction, heart failure, non-lethal heart attack, lethal heart attack, stroke, sudden cardiac death, or a combination thereof.

8. The method of claim 1, wherein the cardiovascular event is development of atherosclerotic cardiovascular disease (ASCVD); the method further comprising:
estimating, using the computational processing system, a risk of ASCVD of the individual via the pooled cohort equation (PCE); and
combining, using the computational processor, the estimated risk of ASCVD as estimated by PCE risk with the predicted likelihood that the individual is to develop ASCVD as determined by the trained computational model to yield a combined risk assessment.

9. The method of claim 8 further comprising administering a statin to the individual, wherein the individual was estimated to be a low risk of developing ASCVD by PCE and high risk of developing ASCVD by the trained computational model.

10. The method of claim 8 further comprising halting administering of a statin to the individual, wherein the individual was estimated to be a high risk of developing ASCVD by PCE and low risk of developing ASCVD by the trained computational model.

11. The method of claim 1 further comprising:
performing a clinical intervention, clinical monitoring, or a treatment based on a future cardiovascular disease event prediction.

12. The method of claim 1 further comprising:
acquiring, using a set of one or more leads of an electrocardiogram, electrocardiogram data of the individual; and
generating, using a computational processor, the one or more electrocardiogram waveforms utilized within the computational model to predict the risk of that the individual will experience a cardiovascular disease event in the future.

13. An electrocardiogram system for predicting future cardiovascular events of patients, the system comprising:
an electrocardiogram device comprising a set of one or more leads capable of acquiring electrical signals of an individual; and
a computational processing system in communication with the electrocardiogram device, the computational processing system comprising:
a memory comprising:
an application for performing an electrocardiogram; and
an application comprising a trained computational model for predicting future cardiovascular events; and
a processor, wherein the application for performing an electrocardiogram directs the processor to:
collect electrical signals of an individual; and
generate electrocardiogram data, wherein the electrocardiogram data comprises a set of one or more electrocardiogram waveforms;
wherein the application comprising a trained computational model for predicting future cardiovascular events directs the processor to:
obtain the electrocardiogram data; and
predict a risk that an individual will experience a cardiovascular disease event in the future utilizing the set of one or more electrocardiogram waveforms.

14. The system of claim 13, wherein the trained computational model is trained from electrocardiogram data obtained from a cohort of individuals having cardiovascular health records that include a timeline of cardiovascular events after collection of each individual's electrocardiogram data.

15. The system of claim 13, wherein the trained computational model predicts the risk that the individual will experience a cardiovascular disease event that will occur in more than one year.

16. The system of claim 13, wherein the trained computational model predicts the risk that the individual will experience a cardiovascular disease event that will occur within five years.

17. The system of claim 13, wherein the cardiovascular event is development of atherosclerotic cardiovascular disease (ASCVD), infarction, heart failure, non-lethal heart attack, lethal heart attack, stroke, sudden cardiac death, or a combination thereof.

18. The system of claim 13, wherein the computational processing system is housed within a computing device that is in direct association the electrocardiogram device.

19. The system of claim 13, wherein the computational processing system is housed within a computing device that is separate of the electrocardiogram device and obtains the electrocardiogram data via a wireless connection.

20. The system of claim 13, wherein the electrocardiogram device and the computational processing system is housed within a wearable device.
